(19) United States
(12) Patent Application Publication
(10) Pub. No.: US 2015/0065920 A1
(43) Pub. Date: Mar. 5, 2015

(54) FOOT ORTHOTIC SERVICE SYSTEM

(71) Applicant: Homeway Technology Co., LTD., Tainan City (TW)

(72) Inventors: Ming-Chia Hsieh, Tainan City (TW); Chin-Hsing Hsieh, Tainan City (TW)

(73) Assignee: Homeway Technology Co., LTD, Tainan City (TW)

(21) Appl. No.: 14/476,169

(22) Filed: Sep. 3, 2014

(30) Foreign Application Priority Data
Sep. 5, 2013 (TW) 102132012

(51) Int. Cl. A61B 5/00; A61B 5/103; A43B 13/38

(52) U.S. Cl. CPC A61B 5/6807; A43B 13/386; A61B 5/1036
USPC 600/592

(57) ABSTRACT

A foot orthotic service system includes a foot orthotic device including a pressure sensing substrate that is capable of generating at least one signal by sensing pressure exerted thereon, and a management unit that is mounted in the pressure sensing substrate, and that includes a signal processing module and a wireless communication module. The signal processing module receives the signal from the pressure sensing substrate, and processes the signal to generate pressure data that is associated with the pressure sensed by the pressure sensing substrate. The wireless communication module is electrically connected to the signal processing module and is capable of wirelessly transmitting the pressure data.

### CROSS-REFERENCE TO RELATED APPLICATION

This application claims priority of Taiwanese Patent Application No. 102132012, filed on Sep. 5, 2013.

### FIELD OF THE INVENTION

The invention relates to an orthotic service system, more particularly to a foot orthotic service system.

### BACKGROUND OF THE INVENTION

To keep track of an orthotic progress of a user's foot, in addition to the user's name, an initial entry of personal data (such as the user's age, weight, height, etc.) and footprints are obtained and stored in a conventional foot orthotic service system located at a foot orthotic service center, and then subsequent entries are entered into the same system during inspection of the user's foot to form an orthotic history file for the user. However, without the user visiting the foot orthotic service center, the service center is in no way to find out the progress of the user's foot. In other words, the conventional foot orthotic service system is unable to keep a real-time progress report or to monitor closely the progress of the user's foot. Moreover, each time the user returns to the foot orthotic service center for a progress check, the user's name must be manually input into the conventional foot orthotic service system in order to retrieve the orthotic history file associated with the user, which is inconvenient and shows room for improvement.

### SUMMARY OF THE INVENTION

Therefore, the object of the present invention is to provide a foot orthotic service system that can eliminate at least one of the aforesaid drawbacks of the prior art.

According to the present invention, there is provided a foot orthotic service system including a foot orthotic device. The foot orthotic device includes a pressure sensing substrate and a management unit. The pressure sensing substrate is adapted for contact with a user's foot, and is capable of generating at least one signal by sensing pressure exerted thereon. The management unit is mounted in the pressure sensing substrate, and includes a signal processing module and a wireless communication module. The signal processing module is electrically connected to the pressure sensing substrate for receiving the signal therefrom, and processes the signal to generate pressure data that is associated with the pressure sensed by the pressure sensing substrate. The wireless communication module is electrically connected to the signal processing module and is capable of wirelessly transmitting the pressure data.

### BRIEF DESCRIPTION OF THE DRAWINGS

Other features and advantages of the present invention will become apparent in the following detailed description of the embodiments with reference to the accompanying drawings, of which:

FIG. 1 is a schematic perspective view of the first embodiment of a foot orthotic service system according to the present invention;

FIGS. 2 to 4 are block diagrams of parts of the first embodiment;

FIG. 5 is a bottom view of a foot orthotic device of the first embodiment;

FIG. 6 is a partly exploded perspective view of a foot orthotic device of the second embodiment of a foot orthotic service system according to the present invention;

FIG. 7 is a sectional view of the foot orthotic device of the second embodiment;

FIG. 8 is a perspective bottom view of a foot orthotic device of the third embodiment of a foot orthotic service system according to the present invention;

FIG. 9 is a top view of the foot orthotic device of the third embodiment; and

FIG. 10 is a sectional view of the foot orthotic device of the third embodiment.

### DETAILED DESCRIPTION OF THE EMBODIMENTS

Before the present invention is described in greater detail, it should be noted that like elements are denoted by the same reference numerals throughout the disclosure.

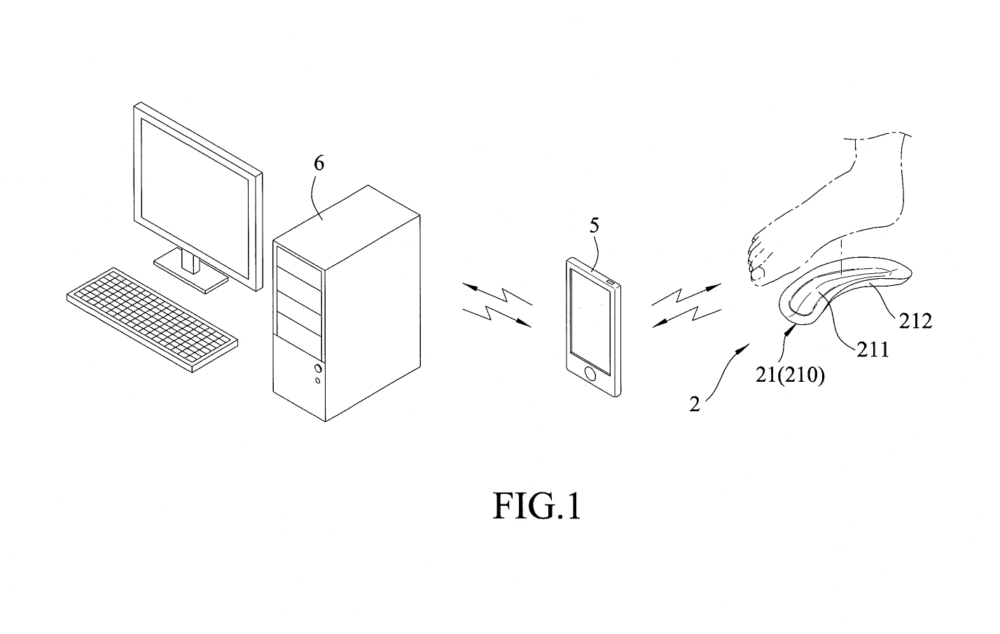

Referring to FIGS. 1, 2, 4 and 5, the first embodiment of a foot orthotic service system includes a foot orthotic device 2, a monitoring device 5, a remote service device 6 and a portable pressure measuring device 7.

The foot orthotic device 2 includes a pressure sensing substrate 21 and a management unit 22. The pressure sensing substrate 21 is adapted for contact with a user's foot, is capable of generating at least one signal by sensing pressure exerted thereon, and includes a main body 210 and at least one sensing member 215. The main body 210 has an orthotic surface 211, an assembling surface 212, a main groove 213 and a plurality of secondary grooves 214. The orthotic surface 211 is used for contact with the user's foot, the assembling surface 212 is opposite to the orthotic surface 211, and the main groove 213 and the secondary grooves 214 are formed in the assembling surface 212. The sensing member 215 is mounted to the assembling surface 212 for generating the signal. In this embodiment, the pressure sensing substrate 21 includes a plurality of spaced-apart sensing members 215.

During actual implementation, the foot orthotic device 2 is disposed in a shoe (not shown) for the user's foot to exert pressure directly onto the orthotic surface 211 of the pressure sensing substrate 21 while engaging in an activity such as jogging or walking, etc., and gradually corrects the user's foot due to the structural design/configuration of the pressure sensing substrate 21.

In this embodiment, each of the sensing members 215 of the pressure sensing substrate 21 is a resistive or capacitive pressure sensor, and is not limited to what is disclosed herein as long as pressure can be measured/sensed. During installation, a waterproofing procedure is conducted to prevent each of the sensing members 215 from being damaged by moisture inside the shoe.

The management unit 22 is mounted in the main groove 213 in the assembling surface 212 of the pressure sensing substrate 21, and includes a signal processing module 221, a memory module 222, a wireless communication module 223 and an identification module 224. The signal processing module 221 is electrically connected to the pressure sensing substrate 21, and specifically to the sensing members 215, for receiving the signal therefrom, and processes the signal(s) to generate pressure data that is associated with the pressure sensed by the pressure sensing substrate 21 (or more specifically, the sensing members 215). The memory module 222 is electrically connected to the signal processing module 221 for electrically storing the pressure data. The wireless communication module 223 is electrically connected to the signal processing module 221 and is capable of wirelessly transmitting the pressure data. The identification module 224 electrically stores information associated with the user of the foot orthotic device 2. In this embodiment, the signal processing module 221 is electrically connected to the sensing members 215 via a plurality of conductive wires 40, each of which is mounted in a respective secondary groove 214. In addition, the wireless communication module 223 is a WIFI communication module in this embodiment and is not limited hereto as long as wireless transmission of data is provided, and the identification module 224 is an RFID (Radio-Frequency Identification) tag. Furthermore, during installation, waterproofing procedure is also conducted to prevent the management unit 22 from being damaged by moisture.

The monitoring device 5 includes a first communication unit 51, a computing unit 53 and an output unit 54. The first communication unit 51 is capable of establishing an electrical connection with the wireless communication module 223 of the management unit 22 of the foot orthotic device 2 for receiving the pressure data therefrom, and is electrically connected to the output unit 54 for transmitting the pressure data thereto to be displayed by the output unit 54. The computing unit 53 is electrically connected to the first communication unit 51 and the output module 54, and generates an alarm signal when the pressure associated with the pressure data exceeds a reference pressure to indicate an anomaly. The output unit 54 is driven by the alarm signal generated by the computing unit 53 to output an alarm. The reference pressure may be set by personnel at the foot orthotic service center, or acquired using other methods. For instance, the monitoring device 5 of this embodiment further includes an analyzing unit 52. The analyzing unit 52 is electrically connected with the first communication unit 51 for receiving pressure data therefrom, and is capable of analyzing multiple entries of the pressure data to generate a reference data that is associated with the reference pressure by, e.g., taking an average of the multiple entries.

In this embodiment, the monitoring device 5 is a smart phone, but may also be a tablet computer, a notebook computer and other portable electronic devices in other embodiments, and is not limited hereto as long as the device can be configured to perform the computation/processing described above and is capable of transmitting data. In addition, the output unit 54 may be a screen and/or a speaker so as to output the alarm in form of an alert message and/or sound notification.

The remote service device 6 is a personal computer or a server, and includes an input unit 61, a storage unit 62, a second communication unit 63, a processing unit 64 and a display unit 65. The input unit 61 is electrically connected to the storage unit 62 for input of, e.g., personal information to establish an orthotic history file associated with the user. The storage unit 62 is used for electrically storing the orthotic history file that is associated with the user. The second communication unit 63 is capable of establishing an electrical connection with the first communication unit 51 of the monitoring device 5 for receiving the pressure data therefrom. Alternatively, the second communication unit 63 is capable of establishing an electrical connection with the wireless communication module 223 of the foot orthotic deice 2 for directly receiving the pressure data therefrom. The processing unit 64, which is a micro-processor in this embodiment, is electrically connected to the second communication unit 63 for receiving the pressure data, and is electrically connected to the storage unit 62 for updating the orthotic history file stored therein with the pressure data. The display unit 65 is electrically connected to the processing unit 64 for displaying the orthotic history files of the user.

The portable pressure measuring device 7 is capable of establishing an electrical connection with the first communication unit 51 of the monitoring device 5 in a wired or wireless manner.

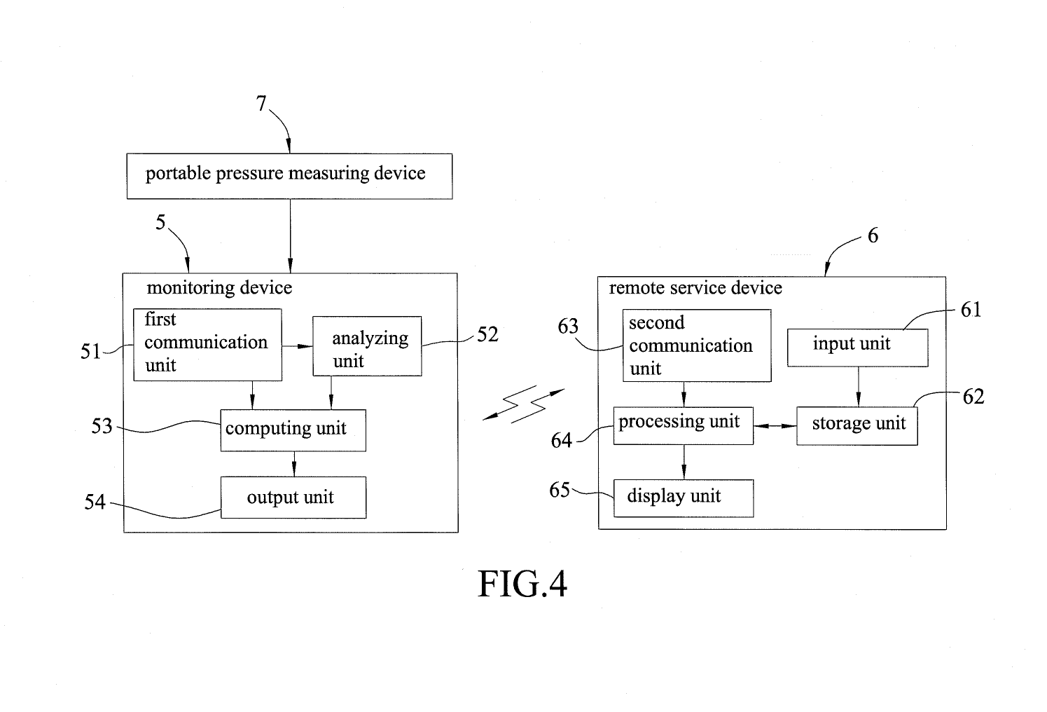

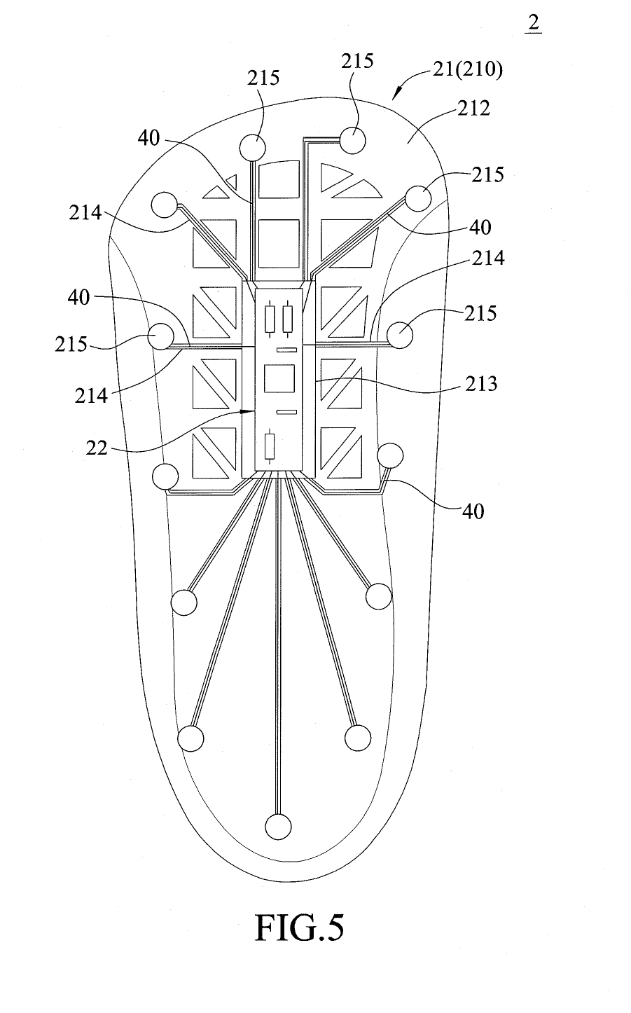

Referring to FIGS. 4 and 5, an initial footprint of the user is obtained to facilitate customizing the foot orthotic device 2 to the user's foot condition and or shape, as well as to incorporate information associated with the user into the foot orthotic device 2, e.g., storing the information in the identification module 24. The information may be the user's ID number, name and/or the like, but is not limited hereto, and may further include the user's age, weight, height, etc. The initial footprint and the information are input via the input unit 61 of the remote service device 6 to be stored in the storage unit 62 as a first entry of the orthotic history file associated with the user. The information in the identification module 224 and in the storage unit 62 can be overwritten to provide accuracy. The foot orthotic service center generally serves multiple clients. Therefore, the storage unit 62 has stored therein a plurality of orthotic history files that are respectively associated with a plurality of users.

The personnel at the foot orthotic service center will then carry the portable pressure measuring device 7 and have the user step thereon to measure and obtain the user's foot's pressure information. Then, the portable pressure measure device 7 transmits the pressure information to the monitoring device 5, which maybe carried by the personnel or the user, using wired or wireless communication. Afterward, the first communication unit 51 of the monitoring device 5 transmits the pressure information to the second communication unit 63 of the remote service device 6 using wired or wireless communication, so that the processing unit 64 can process the pressure information and store it in the orthotic history file, as part of the corrective process.

It should be noted that, since the pressure sensing substrate 21 is shaped according to the user's foot, the pressure information can be used as reference for the design and manufacture of the pressure sensing substrate 21.

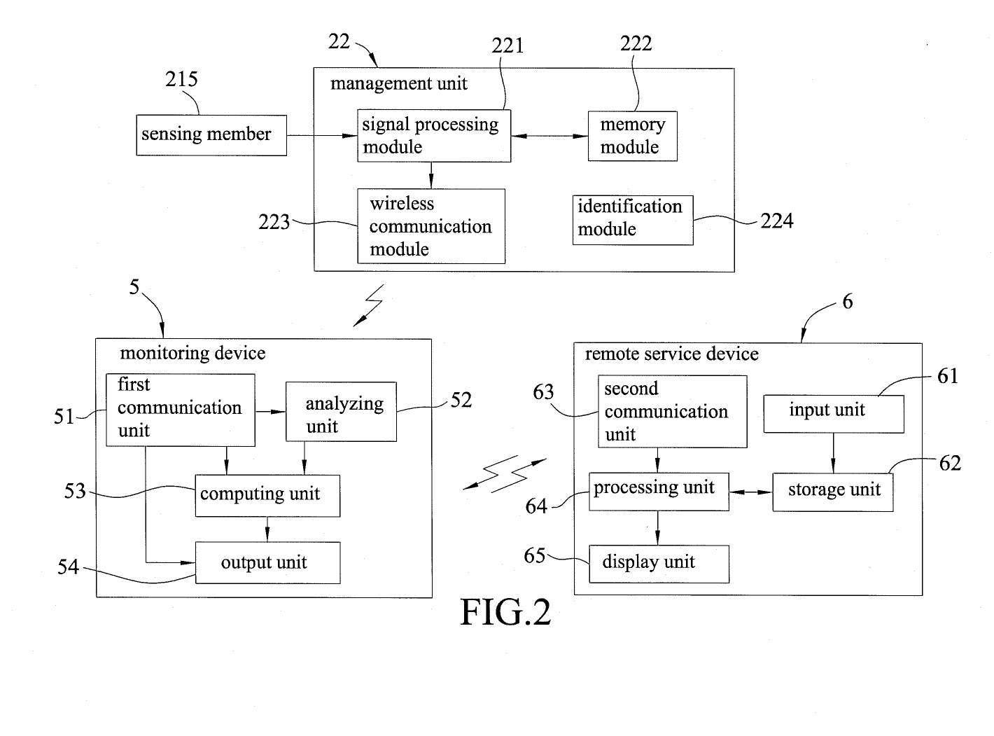

Referring to FIGS. 2 and 5, when the user steps on the foot orthotic device 2, each of the sensing members 215 will sense the pressure exerted by the user's foot at the respective location and generates a signal. The signal processing module 221 of the management unit 22 processes the signal of each of the sensing members 215 and generates pressure data. The wireless communication module 223 then wirelessly transmits the pressure data, which is also stored in the memory module 222.

If the user wishes to check the current plantar pressure or the latest foot condition, the user install vendor-supplied software or an APP in the monitoring device 5 and use, and use it to have the first communication unit 51 receive the pressure data transmitted by the wireless communication module 223, and have the pressure data processed by the computing unit 53 to be outputted by the output unit 54.

If abnormal foot pressure occurs, that is, when the current pressure associated with the current pressure data exceeds the reference pressure, the computing unit 53 will generate an alarm signal which is output by the output unit 54.

During use of the foot orthotic device 2 by the user, the first communication unit 51 of the monitoring device 5 will continue to transmit the most recent pressure data to the second communication unit 63 of the remote service device 6. The processing unit 64 of the remote service device 6 can update the orthotic history file with the newly received pressure data, allowing the personnel at the foot orthotic service center to obtain the latest foot condition of the user.

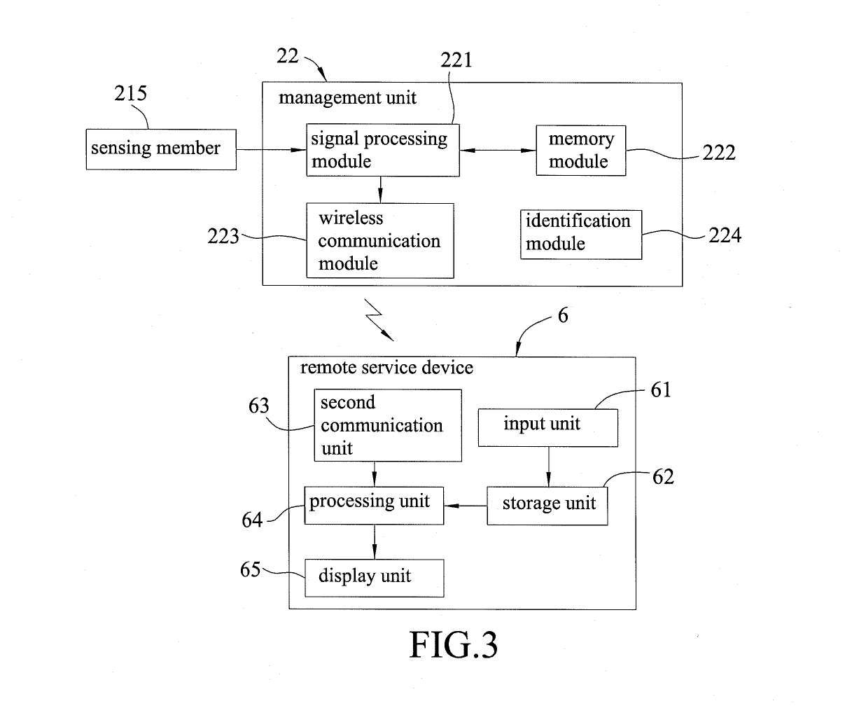

Referring to FIGS. 3 to 5, it is worth mentioning that, after the user has used the foot orthotic device 2 for a period of time, the user returns to the foot orthotic service center for progress inspection and foot orthotic device 2 tuning. The user only needs to place the foot orthotic device 2 proximate to the remote service device 6, so that the second communication unit 63 can read the identification information from the identification module 224 of the management unit 22 of the foot orthotic device 2. The processing unit 64 further receives the identification information from the second communication unit 63 and retrieves from the storage unit 62, based on the identification information, one of the orthotic history files that is associated with the user with which the identification information is associated, and displays it via the display unit 65. Since the orthotic history file is always updated, the personnel can quickly learn the user's current foot condition and proceed to perform progress inspection and foot orthotic device 2 tuning.

Furthermore, before tuning/adjusting, the user can again step on the portable pressure measuring device 7 to generate new pressure information that can be used for the processing unit 64 of the remote service device 6 to compare with the most recent pressure data stored in the orthotic history file and determine whether pressure sensing functionality of the pressure sensing substrate 21 is defective. This allows for simple detection of failure of the sensing members 215 of the foot orthotic device 2.

In sum, the advantages of the present invention are as follows.
1) With the provision of the sensing members 215 and the management unit 22 in the pressure sensing substrate 21, the user can instantly view/check the changes in his/her plantar pressure via the monitoring device 5, allowing the user to be more involved in the correction process.
2) When abnormal conditions are present in the plantar pressure, the monitoring device 5 will immediately generate an alarm to alert the user.
3) The remote service device 6 continuously receives pressure data and updates the orthotic history file, aside from allowing the foot orthotic service center to obtain and store the user's latest foot condition, every time the user returns to the foot orthotic service center, the user only needs to place the foot orthotic device 2 proximate to the remote service device 6 for the remote service device 6 to automatically search for and retrieve the orthotic history file associated with the user to whom the foot orthotic device 2 belongs such that personnel at the foot orthotic service center is immediately informed of the user's current foot condition, thereby enhancing service quality and speed.
4) The pressure information acquired by the portable pressure measuring device 7 may be used as a reference for manufacturing and adjusting the pressure sensing substrate 21. On the other hand, by comparing the pressure information with the pressure data from the foot orthotic device 2, malfunction of any of the sensing members 215 can be detected.

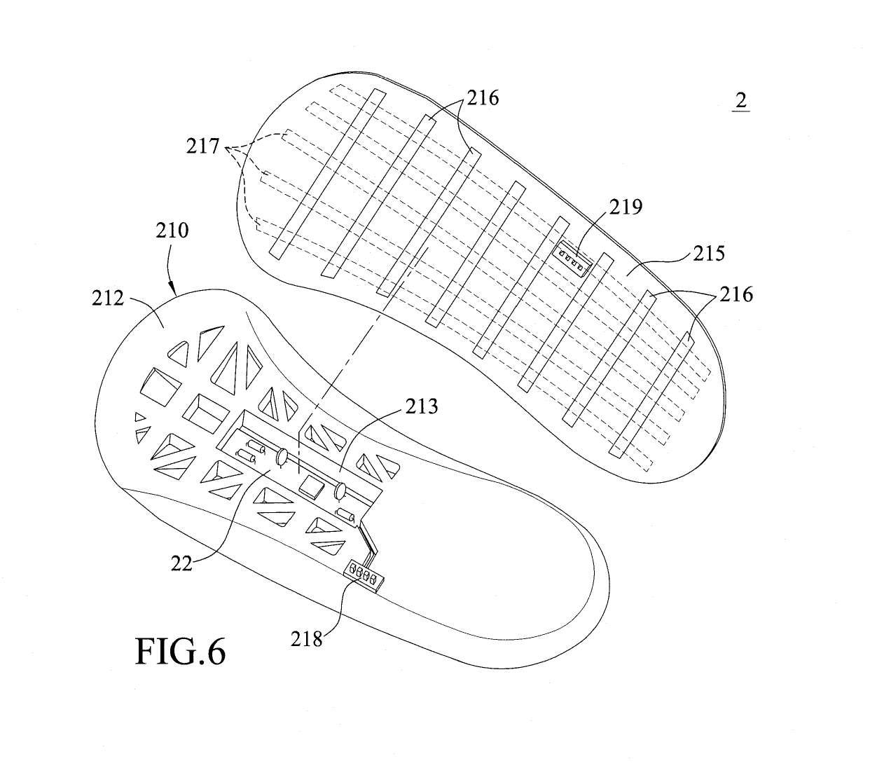

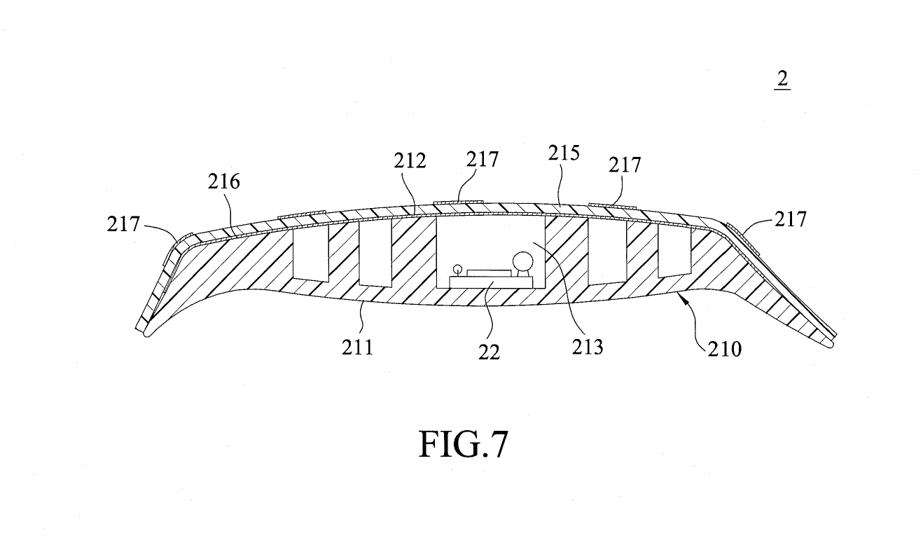

Referring to FIGS. 6 and 7, the second embodiment of the foot orthotic service system according to the present invention is similar to the first embodiment, and only differs in that: the pressure sensing substrate 21 further includes a main body 210 having an orthotic surface 211 and an assembling surface 212, a sensing member 215 in the form of a sheet and having a top surface that is in contact with the assembling surface 212 of the main body 210, and a bottom surface that is opposite to the top surface and that is adapted to contact the inside of the shoe, a plurality of first conductive lines 216 mounted on the top surface of the sensing member 215, and a plurality of second conductive lines 217 mounted on the bottom surface of the sensing member 215. The sensing member 215 may be a capacitive or resistive film pressure sensor, which has a variable capacitance or resistance that varies in response to change in pressure experienced. The pressure sensing substrate 21 further includes a male connector 218 mounted on the assembling surface 212 of the main body 210 and electrically connected to the management unit 22, and a female connector 219 mounted on the top surface of the sensing member 215 and engaged with and electrically connected to the male connector 218. Electrical signals passing through the first and second conductive lines 216, 217 are transmitted to the management unit 22 via the male and female connectors 218, 219. The second embodiment achieves the same effect as the first embodiment.

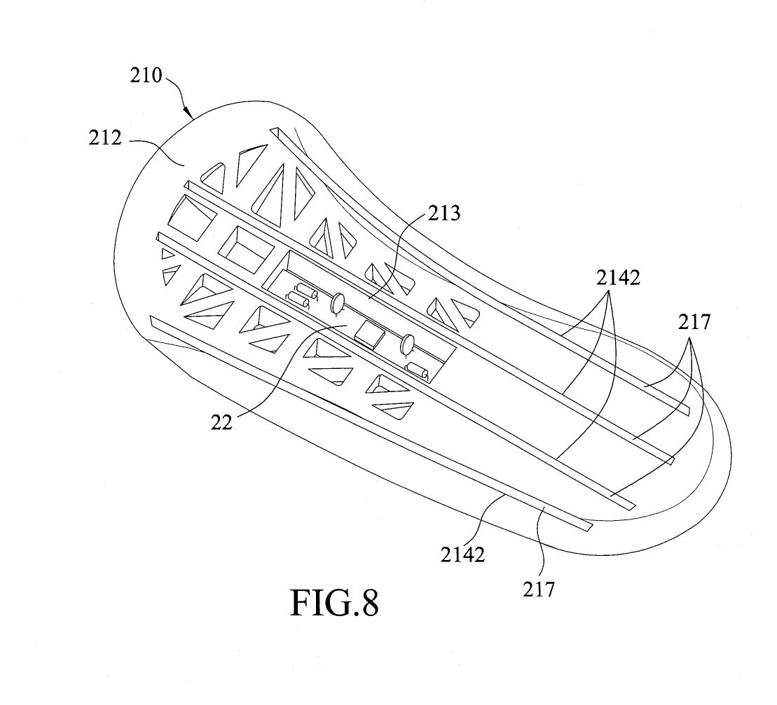

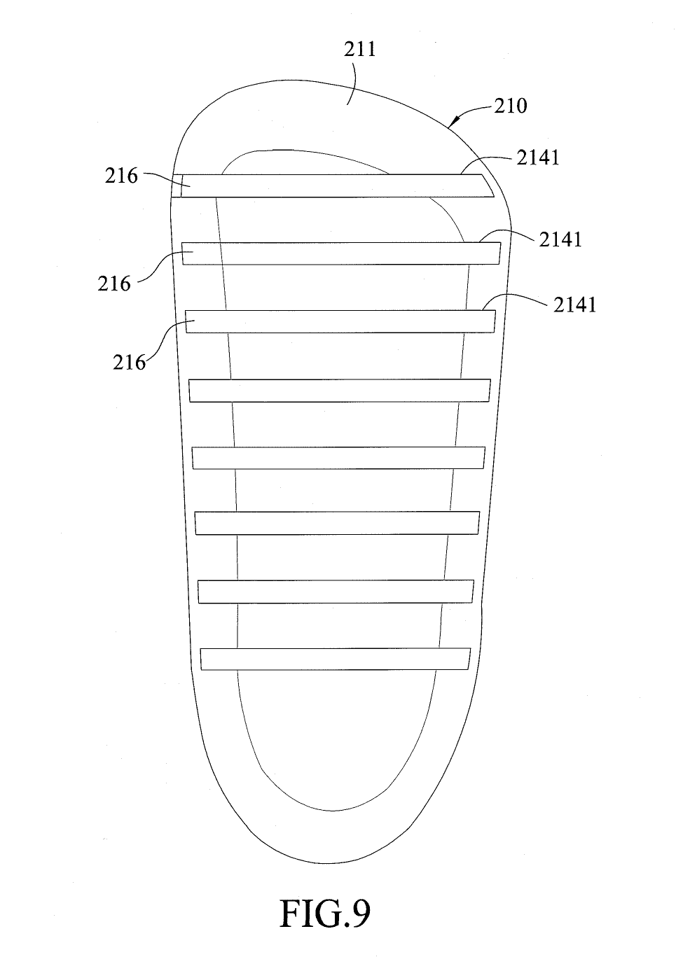

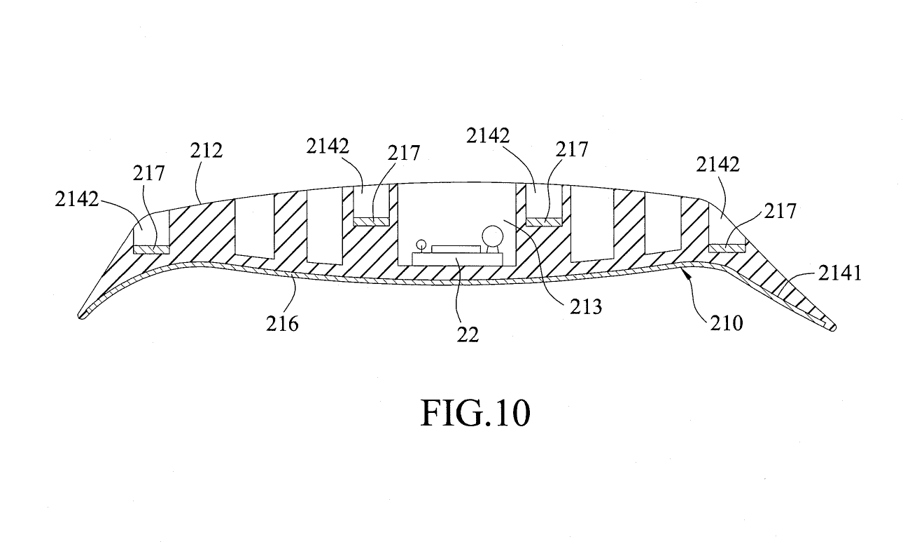

Referring to FIGS. 8 to 10, the third embodiment of the foot orthotic service system according to the present invention is similar to the first embodiment and only differs in the design of the pressure sensing substrate 21.

Contrary to the first embodiment, the pressure sensing substrate 21 of the second embodiment does not include any sensing members 215. Instead, the main body 210 of the pressure sensing substrate 21 is made of a dielectric material, preferably, the high-dielectric Barium Titanate (BaTiO₃), Barium Strontium Titanate (BaSrTiO₃), Strontium Titanate (SrTiO₃) and temperature-stable ceramic capacitor materials (NBC), or a combination thereof to form higher capacitance value, and is formed with, in addition to the main groove 213, a plurality of first secondary grooves 2141 in the orthotic surface 211 and a plurality of second secondary grooves 2142 in the assembling surface 212. In addition, the pressure sensing substrate 21 further includes a plurality of first conductive lines 216, each of which is disposed in corresponding respective first secondary groove 2141, and a plurality of second conductive lines 217, each of which is disposed in corresponding respective second secondary groove 2142. This way, the first and second conductive lines 216, 217 cooperate with the dielectric material of the main body 210 to form a capacitive pressure sensing device for generating the signal.

Alternatively, the first conductive lines 216 may be directly attached to the orthotic surface 211, and the second conductive lines 217 may be directly attached to the assembling surface 212. In other words, the first secondary grooves 2141 and the second secondary grooves 2142 are not necessary in order to achieve the same effect. Furthermore, in practice, the foot orthotic device 2 may be formed with a plurality of through holes (not shown) extending through the orthotic and assembling surfaces 211, 212, for wires (not shown) between the first and second conductive lines 216, 217 allow electric connection therebetween.

While the present invention has been described in connection with what are considered the most practical embodiments, it is understood that this invention is not limited to the disclosed embodiments but is intended to cover various arrangements included within the spirit and scope of the broadest interpretation so as to encompass all such modifications and equivalent arrangements.

What is claimed is:

1. A foot orthotic service system comprising:
a foot orthotic device including
a pressure sensing substrate that is adapted for contact with a user's foot, and that is capable of generating at least one signal by sensing pressure exerted thereon, and
a management unit that is mounted in said pressure sensing substrate, and that includes
a signal processing module electrically connected to said pressure sensing substrate for receiving the signal therefrom, and processing the signal to generate pressure data that is associated with the pressure sensed by said pressure sensing substrate, and
a wireless communication module electrically connected to said signal processing module and capable of wirelessly transmitting the pressure data.

2. The foot orthotic service system as claimed in claim 1, wherein said pressure sensing substrate includes a main body that has
an orthotic surface for contact with the user's foot,
an assembling surface opposite to said orthotic surface, and
a main groove formed in said assembling surface, said management unit being mounted in said main groove.

3. The foot orthotic service system as claimed in claim 2, wherein said pressure sensing substrate further includes at least one sensing member mounted to said assembling surface of said main body for generating the signal.

4. The foot orthotic service system as claimed in claim 2, wherein said main body is made of a dielectric material, and said pressure sensing substrate further includes a plurality of first conductive lines mounted on said orthotic surface of said main body, and a plurality of second conductive lines mounted on said assembling surface of said main body, said first and second conductive lines and said main body cooperatively constituting a capacitive pressure sensing device for generating the signal.

5. The foot orthotic service system as claimed in claim 1, further comprising a monitoring device, said monitoring device including an output unit, and a first communication unit that is capable of establishing an electrical connection with said wireless communication module of said management unit of said foot orthotic device for receiving the pressure data therefrom, and that is electrically connected to said output unit for transmitting the pressure data thereto to be outputted by said output unit.

6. The foot orthotic service system as claimed in claim 5, further comprising a remote service device that includes:
a storage unit for storing an orthotic history file that is associated with the user;
a second communication unit capable of establishing an electrical connection with one of said wireless communication module of said foot orthotic device and said first communication unit of said monitoring device for receiving the pressure data therefrom; and
a processing unit electrically connected to said second communication unit for receiving the pressure data therefrom, and to said storage unit for updating the orthotic history file stored therein with the pressure data.

7. The foot orthotic service system as claimed in claim 6, wherein said storage unit of said remote service device has stored there in a plurality of orthotic history files that are respectively associated with a plurality of users,
said management unit of said foot orthotic device further including an identification module that electrically stores identification information associated with the user of said foot orthotic device,
said second communication unit of said remote service device being capable of reading the identification information from said identification module,
said processing unit of said remote service device further receiving the identification information from said second communication unit, and being capable of retrieving, based on the identification information, one of the orthotic history files that is associated with the user with which the identification information is associated.

8. The foot orthotic service system as claimed in claim 7, wherein said remote service device further includes a display unit that is electrically connected to said processing unit, and that is driven by said processing unit to display the orthotic history file thus retrieved by said processing unit.

9. The foot orthotic service system as claimed in claim 6, wherein said remote service device further includes an input unit electrically connected to said storage unit for input of the orthotic history file.

10. The foot orthotic service system as claimed in claim 6, further comprising a portable pressure measuring device that is capable of generating pressure information by measuring pressure exerted thereon, and that is capable of establishing an electrical connection with said first communication unit of said monitoring device for transmitting the pressure information thereto,
said second communication unit of said remote service device further receiving the pressure information from the first communication module,
said processing unit of said remote service device further receiving the pressure information from said second communication unit, and further updating the orthotic history file with the pressure information.

11. The foot orthotic system as claimed in claim 10, wherein said processing unit of said remote service device is capable of determining whether pressure sensing functionality of said pressure sensing substrate is defective based on the pressure information and the pressure data.

12. The foot orthotic service system as claimed in claim 5, wherein said monitoring device further includes a computing unit that is electrically connected to said first communication unit and said output unit, and that generates an alarm signal when the pressure associated with the pressure data exceeds a reference pressure;
said output unit being driven by the alarm signal from said computing unit to output an alarm.

13. The foot orthotic system as claimed in claim 12, wherein said monitoring device further includes an analyzing unit that is electrically connected with said first communication unit for receiving the pressure data therefrom, and that is capable of analyzing multiple entries of the pressure data to generate a reference data that is associated with the reference pressure.
